(19) United States
(12) Patent Application Publication
(10) Pub. No.: US 2026/0224123 A1
(43) Pub. Date: Aug. 6, 2026

(54) DEVICE AND METHOD FOR EXTERNALLY MEASURING BLOOD FLOW

(71) Applicants: Agency for Science, Technology and Research, Singapore (SG); National University of Singapore, Singapore (SG); National University Hospital (Singapore) Pte Ltd, Singapore (SG)

(72) Inventors: Renzhe Bi, Singapore (SG); Pei Jackie Ho, Singapore (SG); Ruochong Zhang, Singapore (SG); Yi Qi, Singapore (SG); Lingyan Meng, Singapore (SG); Malini Olivo, Singapore (SG)

(21) Appl. No.: 19/155,057

(22) PCT Filed: Feb. 7, 2024
(86) PCT No.: PCT/SG2024/050064

§ 371 Date: Aug. 8, 2025

(30) Foreign Application Priority Data
Feb. 9, 2023 (SG) 10202300322X

(51) Int. Cl. A61B 5/026; A61B 5/024

(52) U.S. Cl. CPC A61B 5/0261; A61B 5/02416; A61B 5/02438

(57) ABSTRACT

A sensing device, a wearable device, and a method of use to externally measure blood flow is described. The sensing device includes a single printed circuit board, one or more laser diodes configured to emit light at an object, one or more a laser drivers configured to supply power to the one or more laser diodes, an image sensor configured to receive the light reflected and/or scattered from the object, and a controller configured to process a signal from the image sensor, the signal being related to the received light, wherein the one or more laser diodes, the one or more laser drivers, the image sensor, and the controller are integrally disposed on the single printed circuit board. The wearable device includes the sensing device and a fastening mechanism. The sensing device and wearable device may be used to externally measure blood flow.

### REFERENCE TO RELATED APPLICATIONS

The present application claims priority to Singapore patent application number 10202300322X with a filing date of 9 Feb. 2023 and titled “A Wearable Sensor For Haemodynamic Sensing”

### FIELD OF THE INVENTION

The present application relates to a device and method for externally measuring blood flow by the emission of light at an object and detection of reflected light from the object.

### BACKGROUND OF THE INVENTION

Typical haemodynamic sensing technologies, such as laser Doppler flowmetry, laser speckle contrast imaging, spatial frequency domain imaging, diffuse correlation spectroscopy system require a device console and a fibre-based sensor or camera with an imaging lens to measure the flow or the dynamics of scattering liquid. However, the size of these devices is relatively big and is not portable. Optical fibres are used in blood flow measurement devices because typical long coherence lasers are large in size and the typical detectors like a CCD camera is also bulky. Therefore, optical fibres are used to deliver and collect signals in between the device console and human body, and makes its use as a wearable device impractical.

Wearable devices are increasingly popular in both lifestyle and medical applications as they are portable and can be readily worn by a user during his/her daily activities. Haemodynamic waveform carries rich physiological information, such as blood vessel stiffness, blood pressure and tissue pulsation. Thus, a wearable device that is able to perform haemodynamic waveform measurement may be readily used by users to monitor their physical wellbeing and seek medical attention early.

However, existing wearable devices generally contain only the sensing part, in particular for haemodynamic waveform measurement, but not the computational unit. This means that an external laptop or desktop is still needed to perform the signal processing and computation of the information obtained from the sensing part. The external computer is thus not considered to be integrally disposed with the sensing component. Existing devices have two main challenges, namely to reduce their sizes to make them small enough to be wearable devices, and to cater for the size of a laser and the high computation power required of the processor.

Similar modalities use relatively bulky laser modules with higher stability as a small size laser driver is normally not stable enough to produce a coherent laser. To achieve a wearable size for the proposed sensing device, the components of the laser module and drive circuit need to be simplified as much as possible. However, a new challenge arises to examine whether the miniaturised laser module has sufficient coherence length as traditional methods are either expensive or difficult to use. Moreover, all existing coherence length measurements are not directly relevant to the usability of the laser module in haemodynamic waveform measurement. As a result, the miniaturisation of the laser module poses a technical problem. Furthermore, existing controllers (or processors) are generally required to be very large to possess high and sufficient computing power to handle the complex calculations like background noise removal and calculation of the autocorrelation of the detected signals. As a result, processors of such existing devices cannot fit into a reasonable size of a wearable device.

It would be appreciated that a reasonable size of the wearable device is relative, but it will be desirable to have a controller and hence wearable device as small and light as possible to allow the user to wear the wearable device for an extended period of time. For example, while some people may strap their mobile device around their arm while running or exercising for a short period of time, this is not practical or feasible for most people for extended use that a wearable device is intended for. Small sized controllers (or microcontrollers (MCUs) normally used in wearable devices cannot provide sufficient computational power required for existing devices.

### SUMMARY OF THE INVENTION

In a first aspect, there is provided a sensing device comprising a single printed circuit board; one or more laser diodes configured to emit light at an object; one or more laser drivers configured to supply power to the one or more laser diodes; an image sensor configured to receive the light reflected and/or scattered from the object; and a controller configured to process a signal from the image sensor, the signal being related to the received light, wherein the one or more laser diodes, the laser driver, the image sensor, and the controller are integrally disposed on the single printed circuit board. One laser driver may be used to supply power to one laser diode, multiple (i.e. two or more) or all the laser diodes and may be configured as required.

In an embodiment, the image sensor comprises a complementary metal-oxide-semiconductor (CMOS) sensor or a charge-couple device (CCD) sensor.

In an embodiment, the image sensor is configured to operate without an optical conduit to receive the light reflected and/or scattered from the object.

In an embodiment, the signal comprises a plurality of laser speckle patterns, and wherein the controller is configured to process the signal to measure changes in the plurality of laser speckle patterns. In an embodiment, the controller comprises a micro system on module or a field-programmable gate array.

In an embodiment, the sensing device is operable when in contact or in proximity to a surface of the object and the object is a user.

In an embodiment, the image sensor is configured to capture the light reflected and/or scattered from the object as a plurality of images at a sampling rate of at least 100 Hz, preferably from 100 Hz to 500 Hz.

In an embodiment, each of the plurality of images comprises a plurality of pixels at each timepoint, the controller being configured to determine a Dynamics index at each timepoint, wherein the Dynamics index is calculated as

$${( \frac{I}{S . D .} )}^{x} ,$$

wherein I is the mean intensity of the pixels, S.D. is the standard deviation of the plurality of pixels, and x is from 1.8 to 2.6, preferably x is 2.2.

In an embodiment, the plurality of pixels comprises an imaging field at a center region of the image sensor, the imaging field having a range from 7×7 pixels to 300×300 pixels.

In an embodiment, the controller is configured to generate a plurality of the Dynamics indices at different timepoints based on the sampling rate; to generate a waveform based on the plurality of Dynamics indices; and to convert the waveform into a frequency domain.

In an embodiment, the controller is configured to determine a ratio of a sum of amplitude values at a heartbeat frequency range in the frequency domain to a sum of amplitude values at a baseline frequency range in the frequency domain.

In an embodiment, the heartbeat frequency range comprises from a heartbeat frequency minus 0.1 Hz to the heartbeat frequency plus 0.1 Hz and the baseline frequency range is from 0 Hz to 0.5 Hz. Preferably, the controller is configured to determine a ratio of an amplitude of a pulse rate frequency to an amplitude of frequency zero, or a square root of the ratio of the amplitude of the pulse rate frequency to the amplitude of frequency zero.

In an embodiment, the controller is configured to determine the Dynamics index without removal of background noise in the signal.

Preferably, the sensing device further comprises a display unit configured to display the ratio, or an indication based on the ratio.

In an embodiment, a distance between the one or more laser diodes and the image sensor is at least twice a penetration depth of the light into the object.

In an embodiment, the sensing device further comprises at least one of the following: a transmitter, a receiver, and a transceiver, wherein the transmitter, the receiver, and/or the transceiver is coupled to the controller and is configured to communicate wirelessly with an external device. In an embodiment, the sensing device further comprises a power unit configured to provide electrical power to the sensing device.

In an embodiment, the sensing device comprises a plurality of laser drivers, each laser driver is configured to supply power to one laser diode or multiple laser diodes. In an embodiment, the sensing device comprises a plurality of laser diodes.

In an embodiment, the one or more laser diodes each operates at a low optical power of 1 mW to 10 mW and the laser driver is a constant current source.

Preferably, an operating wavelength of each of the one or more laser diodes is from 450 nm to 1300 nm, preferably from 700 nm to 950 nm.

Preferably, each of the one or more laser diodes has a narrow linewidth of less than 0.1 mm.

In a second aspect, there is provided a wearable device comprising the sensing device according to the first aspect; and a fastening mechanism adapted to couple the sensing device to a user. For example, the wearable device comprises a watch, a wristband, or a skin patch to be pasted on a skin of the user.

In a third aspect, there is provided a method of externally measuring blood flow, the method comprising emitting light at an object with blood flow beneath a surface of the object; receiving the light reflected and/or scattered from the object; and processing a signal relating to the received light. The method of the third aspect may be a computer-implemented method, in particular implemented by a controller in the sensing device.

Preferably, the method further comprises capturing the light reflected and/or scattered from the object as a plurality of images at a sampling rate of at least 100 Hz, preferably from 100 Hz to 500 Hz.

Preferably, each of the plurality of images comprises a plurality of pixels at each timepoint, and the method further comprises determining a Dynamics index at each timepoint, wherein the Dynamics index is calculated as

$${( \frac{I}{S . D .} )}^{x} ,$$

I being the mean intensity of the pixels, S.D. being the standard deviation of the plurality of pixels, and x being from 1.8 to 2.6, preferably x being 2.2.

Preferably, the method further comprises generating a plurality of the Dynamic indices at different timepoints based on the sampling rate; generating a waveform based on the plurality of Dynamics indices; and converting the waveform into a frequency domain.

Preferably, the method further comprises determining a ratio of a sum of amplitude values at a heartbeat frequency range in the frequency domain to a sum of amplitude values at a baseline frequency range in the frequency domain.

Preferably, the heartbeat frequency range comprises from a heartbeat frequency minus 0.1 Hz to the heartbeat frequency plus 0.1 Hz and preferably the baseline frequency range is from 0 to 0.5 Hz.

Preferably, the method further comprises determining a ratio of an amplitude of a pulse rate frequency to an amplitude of frequency zero, or a square root of the ratio of the amplitude of the pulse rate frequency to the amplitude of frequency zero.

Preferably, determining the Dynamics index is performed without removal of background noise of the signal.

Preferably, the method is performed by the sensing device according to any one of claims 1 to 21 or the wearable device according to claim 22 or 23.

In a fourth aspect, there is provided a non-transitory computer readable medium comprising instructions that, when executed on a processor, perform the method according to the third aspect.

In a fifth aspect, there is provided a method of determining viability of a tissue in a subject, the method comprising obtaining a first ratio of a sum of amplitude values at a heartbeat frequency range to a sum of amplitude values at a baseline frequency range or a second ratio of an amplitude of a pulse rate frequency to an amplitude of frequency zero, preferably a square root of the second ratio; and determining the viability of the tissue based on the first and/or the second ratio. Preferably, the first ratio is obtained by the methods according to the third aspect.

In a sixth aspect, there is provided a method of determining usability of a laser module in haemodynamic waveform measurement, the method comprising emitting light from the laser module at a rough surface of a metal; receiving the light reflected from the rough surface to capture a plurality of images over a period of time, wherein each of the plurality of images comprises a plurality of pixels at each timepoint; and determining a Dynamics index at each timepoint over the period of time, wherein the Dynamics index is calculated as

$${( \frac{I}{S . D .} )}^{x} ,$$

wherein I is the mean intensity of the pixels, S.D. is the standard deviation of the plurality of pixels, and x is from 1.8 to 2.6, preferably x is 2.2. Preferably, the method further comprises determining the laser module to be usable when the Dynamic index is determined to be from 0 to 20 with a standard deviation of less than 2.

Advantageously, the sensing device may be used to emit light into deep tissue beneath the surface of a user and measure the properties of the deep tissue. For example, the sensing device may be used to measure blood flow parameters and a hemodynamic waveform from the measurements.

Advantageously, the sensing device may be incorporated into a wearable device by the reduction in the size of the various components including the miniaturisation of the laser module and reduction of the computation power required by the controller in the sensing device. The reduction of the computation power is achieved by avoiding the use of complicated mathematical functions and the removal of background noise. The incorporation of the sensing device into a wearable device that is small and light allows users to routinely monitor their personal health and seek medical attention early if necessary.

### BRIEF DESCRIPTION OF THE DRA WINGS

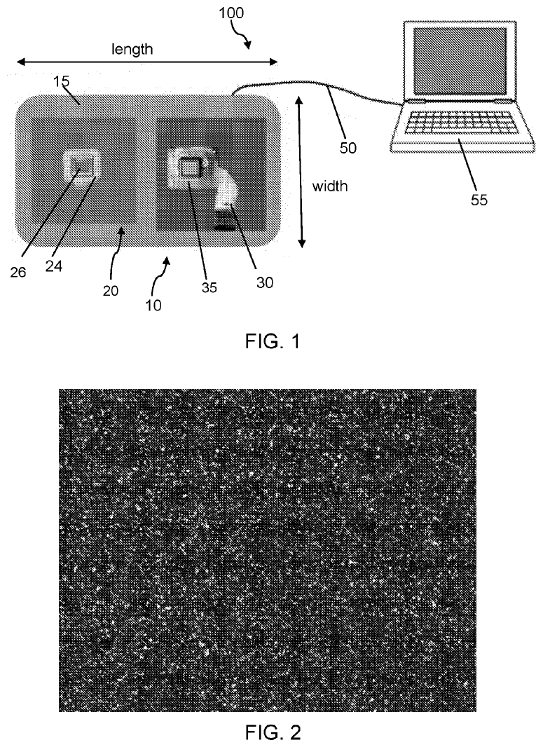

FIG. 1 shows a schematic representation illustrating a concept design of a device for externally measuring blood flow.

FIG. 2 shows an image of patterns that are captured by a CMOS sensor, according to an example.

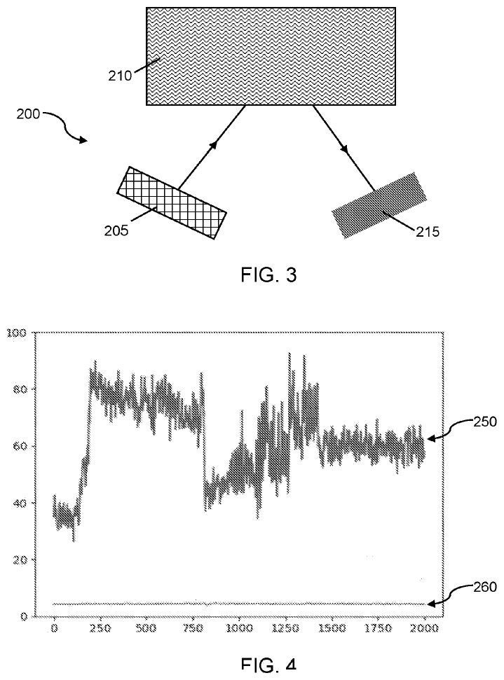

FIG. 3 shows a schematic representation illustrating a setup for examining and determining whether a laser module is sufficiently coherent for haemodynamic waveform measurement, according to various embodiments.

FIG. 4 shows a graph illustrating examples of readings from a qualified laser module and a disqualified laser module.

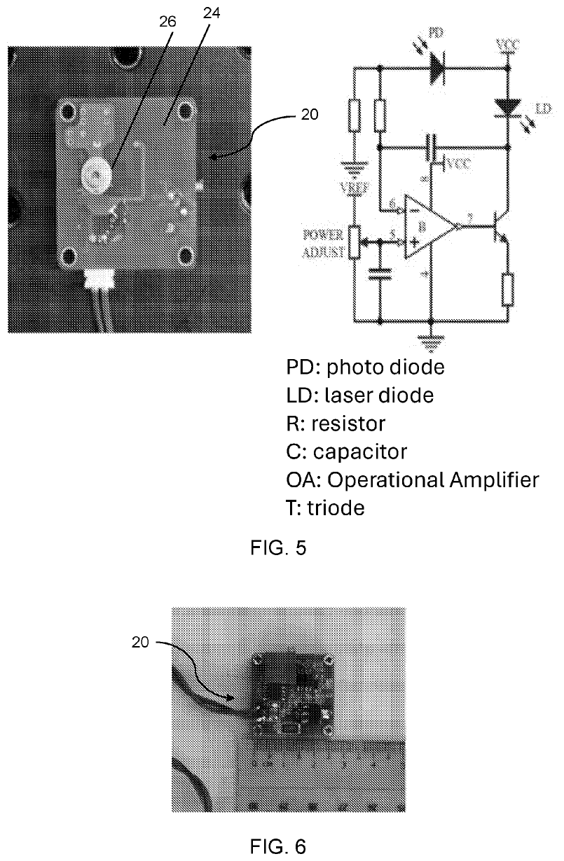

FIG. 5 shows a photograph (left) and a schematic circuit representation (right) of an example of an exemplary wearable device with a laser diode soldered on a laser driver board to avoid noise and to reduce the size of the wearable device. A constant current driver with feedback loop is used to drive the laser diode.

FIG. 6 shows a picture of an example of a laser driver measuring approximately 26 mm×26 mm.

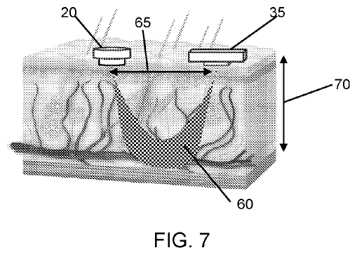

FIG. 7 shows a schematic of an embodiment of the sensing device in use.

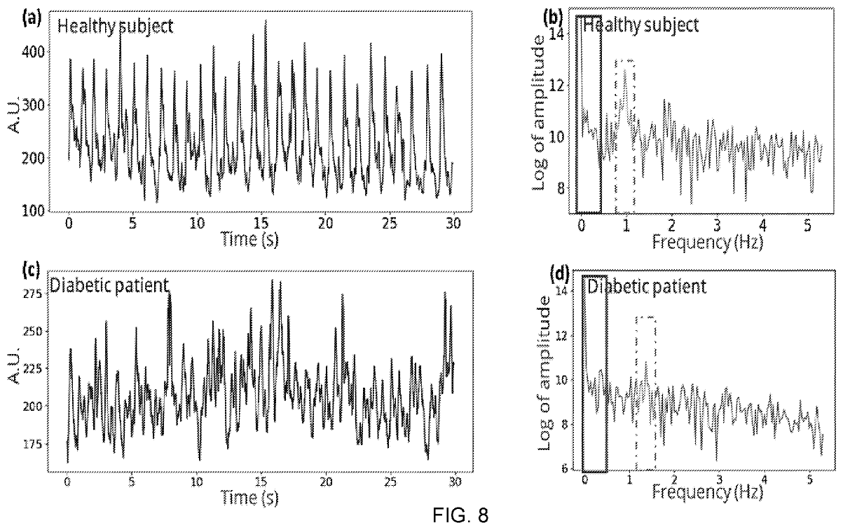

FIG. 8 shows graphs illustrating the comparison of haemodynamic waveform of a healthy subject's foot (Panel a) and a diabetic patient's foot (Panel c), with Panel b and Panel d respectively showing the spectral density of the raw data from Panel a and Panel c, according to various examples.

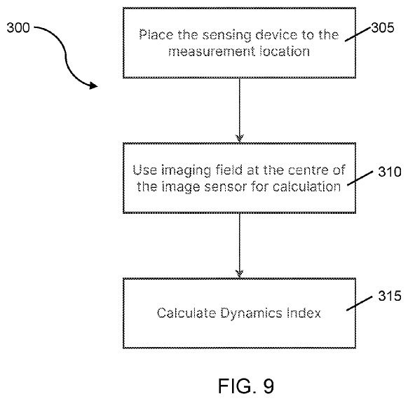

FIG. 9 shows a flow diagram illustrating an example of a method of obtaining sensor information.

### DETAILED DESCRIPTION OF THE INVENTION

The following detailed description is merely exemplary in nature and is not intended to limit the invention or the application and uses of the invention. Furthermore, there is no intention to be bound by any theory presented in the preceding background of the invention or the following detailed description. Other technical advantages may become readily apparent to one of ordinary skill in the art after review of the following figures and description. Embodiments described in the context of one of the methods or devices are analogously valid for the other methods or devices. Similarly, embodiments described in the context of a method are analogously valid for a device, and vice versa.

The terms “about”, “approximately” must be read with reference to the context of the application as a whole, and have regard to the meaning a particular technical term qualified by such a word usually has in the field concerned. For example, it may be understood that a certain parameter, function, effect, or result can be performed or obtained within a certain tolerance, and the skilled person in the relevant technical field knows how to obtain the tolerance of such term. The phrase “at least one of A and B” means it requires only A alone, B alone, or A and B, i.e. only one of A or B is required.

As used herein, the articles “a”, “an” and “the” as used with regard to a feature or element include a reference to one or more of the features or elements. As used herein, the term “and/or” includes any and all combinations of one or more of the associated listed items. As used herein, the terms “first,” “second,” and “third,” etc. are used merely as labels, and are not intended to impose numerical requirements on their objects. As used herein, the terms “top”, “bottom”, “left”, “right”, “side”, “vertical” and “horizontal” are used to describe relative arrangements of the elements and features. As used herein, the term “each other” denotes a reciprocal relation between two or more objects, depending on the number of objects involved.

Terms such as “connected”, and “attached” are used interchangeably herein and encompass direct as well as indirect connection, or attachment unless the context clearly dictates otherwise.

Where a combination is disclosed, each subcombination of the elements of that combination is also specifically disclosed and is within the scope of the invention. Conversely, where different elements or groups of elements are disclosed, combinations thereof are also disclosed. Where any element of an invention is disclosed as having a plurality of alternatives, examples of that invention in which each alternative is excluded singly or in any combination with the other alternatives are also hereby disclosed; more than one element of an invention can have such exclusions, and all combinations of elements having such exclusions are hereby disclosed.

Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the invention. Where a value being discussed has inherent limits, those inherent limits are specifically disclosed. Where a value is explicitly recited, it is to be understood that values which are about the same quantity or amount as the recited value are also within the scope of the invention, as are ranges based thereon.

A standalone functionable wearable device is described herein which does not require a separate or bulky control unit to work. The devices described herein provides haemodynamic waveform measurement with the same quality and results as existing devices and methods using bulky device consoles. Further, the devices described herein are without any optical conduit like optical fibre or lens which considerably reduces the size of the device making it possible to be used as a wearable device while delivering the same result.

A wearable device includes any device that may be placed into close contact with a user's skin to measure the blood flow below or underneath the skin. The wearable device may be worn around the skin or stuck or pasted on the skin. Non-limiting examples of a wearable device includes a watch, a wristband, and a skin patch to be pasted on a skin of the user. The watch or wristband may be worn on any part of an arm of a user or even possibly the leg. The skin patch may be placed at any part of the skin, but presumably where there is blood flow.

The wearable device has a sensing device (or a sensor device or more simply a sensor) 10 and a fastening mechanism adapted to couple the sensing device 10 to the user. The fastening mechanism depends on the wearable device. For example, if the wearable device is a watch or a wrist band, the fastening mechanism may be strap and a clasp like a deployant clasp, a pin buckle, sliding buckle, a Velcro strap, an elastic strap, or any other suitable mechanisms. For a skin patch, the fastening mechanism may be an adhesive.

FIG. 1 shows an embodiment of the sensing device 10. The sensing device 10 may include a single printed circuit board (PCB) 15 with a laser module 20, an image sensor module, and a controller (interchangeably referred to as a processor or computational unit) disposed or attached on the single piece of PCB 15. In other words, the components for the laser module 20, the image sensor module, and the controller are connected to one another (where applicable) via circuitries and electrically conductive tracks provided on the PCB 15. The laser module 20 may include one or more laser diode driver circuits (or laser drivers) 24 and one or more laser diodes 26 that is small and stable. Multiple laser drivers 24 and laser diodes 26 may be used in the same manner as one laser driver 24 and one laser diode 26. The laser diode 26 may be configured to emit light at an object with blood flow beneath a surface of the object, for example the skin of a user, to measure the blood flow. The laser driver 24 may be configured to supply power to the laser diode 26. If multiple laser diodes 26 present, one laser driver 24 may be used to supply power to all the laser diodes 26. Alternatively, two or more laser drivers 24 may be provided and each laser driver 24 may be connected to one or more of the laser diodes 26 in any suitable and desired configuration. The image sensor module may include an image sensor 35 and the image sensor's driver circuit 30. The image sensor 35 may be configured to receive the light reflected and/or scattered from the object. Examples of the image sensor 35 that may be used include a complementary metal-oxide-semiconductor (CMOS) sensor or a charge-couple device (CCD) sensor. The image sensor 35 may be connected to the other components of the sensing device 10 by any suitable means. In an embodiment, the image sensor 35 may be a CMOS sensor. The CMOS sensor may be controllable with a miniaturized driver and/or the controller. An example may be a MIPI CSI-2 compatible CMOS sensor. CSI refers to camera serial interface. In an example, the image sensor 35 may be a Universal Serial Bus (USB) compatible CMOS sensor or CCD sensor.

The PCB board 15 may be a rigid PCB 15 or a soft PCB 15 (may be called a flexible PCB). A soft or flexible PCB 15 may be more suited to be used as a wearable device. The PCB 15 supports the various components and electrically connects the components.

The sensing device 10 may contain a power unit configured to provide electrical power to the sensing device 10 such as the components like the laser module, image sensor module and controller. For example, the power unit may contain a battery such as a rechargeable battery or a disposable battery. Since the sensing device 10 has low power requirements, a small battery may be sufficient. Other suitable means to provide the electrical power may also be used. In some embodiments, the battery may be separate from the sensing device 10, i.e. supplied by the wearable device or other external power source or supply, and the power unit may refer to a circuitry to connect an external power supply to the sensing device 10.

The sensing device 10 may have only one piece of PCB 15 that connects the function of the laser 26 and the laser driver 24, a CCD/CMOS sensor as an example of the image sensor 35, and a controller for the CCD/CMOS sensor. Multiple laser diodes 26 and laser drivers 24 may be provided on the single piece of PCB. This enables the laser module 20, image sensor 35 and controller to be integrally disposed on or integrated in the same PCB board 15 and makes the true wearable device format possible. This also enables the signal processing and computation to be performed in the embedded controller without the need for additional external computers. The integral design of the sensing device 10 is in vast contrast to existing sensor devices that utilise a separate or external processing unit (for example, a laptop or a handheld smartphone mobile device). For example, the sensing device 10 containing the single PCB 15 with the integrated laser diode 26, the laser driver 24, the image sensor 35 and the controller may be used in a wearable sensor device for non-invasive haemodynamic waveform measurement. Advantageously, the sensing device 10 now functions as a single unit instead of two units (separate sensor and controller) as in most existing prior art. With this, the size of the wearable device is much smaller than existing devices for haemodynamic waveform measurements. The small size allows the sensing device 10 to be used as a user-friendly and comfortable wearable device that can be freely worn by a user compared to devices with a separate bulky controller.

The entire laser module including the laser diode 26 and laser driver 24 may be engineered to be significantly small and integrated into the sensing device 10. For example, the entire laser module may be dimensioned planarly at about 30 mm by 30 mm (or less) and the image sensor module may also be dimensioned planarly at about 30 mm by 30 mm (or less), giving approximately a length of 60 mm by a width of 30 mm sensing device 10 as shown in FIG. 1 where the two approximately 30 mm by 30 mm modules are placed next to each other. The sensing device 10 may be about 8 mm thick and is thus considered to be very small with dimensions of 60 mm (length)×30 mm (width)×8 mm (thickness). The size of the modules are provided here merely as an example and modules of other dimensions may be generally used as long as it is sufficiently small to form a wearable device. For example, the sensing device 10 may possibly have dimensions of up to 20 cm×10 cm and a thickness of 5 cm or less (preferably 3 cm or less). It will be advantageous for the sensing device 10 to be as small as possible and the physical limitations are mainly due to the components. FIG. 5 shows the laser diode 26 soldered together with the driver board. Advantageously, the laser diode 26, image sensor 35 and controller are integrally disposed on a single PCB board 15 that avoids or at least minimises noise and reduces the size of the wearable device.

In an embodiment, the sensing device 10 may have multiple PCBs with each containing the laser module 20, image sensor 35 and controller and is able to function as the sensing device 10 itself.

In the process of the miniaturisation of the laser module a method to quantitatively examine the usability of the laser modules in haemodynamic waveform measurement was developed. In other words, this method may be for determining whether the laser module has sufficient coherence length for haemodynamic waveform measurement. This method has been verified through several iterations of laser module design and prototyping. An example of the setup is shown in FIG. 3. A laser light is projected, from the laser module 205 being tested, onto an opaque and rough metal surface 210 through a diverging beam, then a CMOS sensor 215 is used to receive the reflected optical signals without any lens or fibres in front of the CMOS sensor 215. The setup in FIG. 3 may be used to collect the reflected light and calculate the Dynamic Index continuously.

The Dynamics index may be defined as:

$$Dynamics index = {( \frac{I}{S . D .} )}^{x} ,$$

where I is the mean intensity of all the pixels, S.D. is the standard deviation of all the pixels, and x is from a range of 1.8 to 2.6. The value of x as 2.2 is an optimal value from theory simulation performed.

The calculated Dynamic indices over a period may be plotted as shown in FIG. 4. It was observed that a qualified laser module should have a stable reading between 0 to 20, with a standard deviation of less than 2 as shown in the near straight line 260 at the bottom of the plotted graph. A typical disqualified laser module will have much bigger fluctuation in the Dynamic index reading as shown in the plotted readings 250 at the top in FIG. 4, although the intensity of the laser could be considered stable. This method is a simple and accurate way to characterise whether the laser diode 26 and sensing device 10 is sufficiently ‘coherent’ for haemodynamic waveform measurement. This method is especially useful to develop a series of laser drivers 24 and laser diodes 26 for testing and screening purpose.

FIG. 6 shows an example of the laser module (laser driver 24 and laser diode 26) with dimensions of approximately 26 mm×26 mm (or approximately 30 mm×30 mm), according to one example. It may be powered by a 3.7 V lithium battery. The optical power required for the sensing device 10 is low (10 mW or less, for example from 1 to 10 mW). However, having such lower optical power may not be able to drive most existing laser diodes in an optimal operating region. In an embodiment, a laser diode 26 with a significantly low current threshold was used. The laser diode 26 can work at low optical power (1-5 mW) with good stability and narrow linewidth (typically less than 0.1 mm). If more power is required, the heating may negatively affect the performance of the sensing device 10 due to the miniaturisation of the sensing device 10. The operating wavelength of the laser module may be from 450 nm to 1300 nm, preferably from 700 nm to 950 nm. In an example, the operating wavelength may be 780 nm. Further, the narrow linewidth provides a more coherent laser. The laser driver 24 may be customised to the required specification, for example to regulate the current and voltage to the laser diode 26 to ensure the low optical power operation is stable and efficient. In an example, the laser driver 24 may be a constant current laser driver 24 (i.e. a constant current source) with a feedback loop to drive the laser diode 26. The laser diode 26 may touch or be in contact with the skin of the user (i.e. the object) or may be in close proximity to the skin. Thus, the laser light does not travel much distance through the air and goes directly into the skin and diffuses through the skin to reach the blood underneath the skin. The light is scattered by the skin layer and the blood flow, and each affects different properties of the reflected light. The methods and sensing device 10 functions by correlating only the laser speckle changes after the scattering and reflection by the blood flow patterns (hemodynamics). Advantageously, this avoids the influence of skin scattering and effects of the light by the skin of different users, and no calibration of the sensing device 10 is required to account for the skin effects. The choice of the laser wavelength also minimises the skin absorption and scattering.

FIG. 7 shows a simplified diagram of how the sensing device 10 operates. The laser module 20 emits the light which penetrates the skin and is scattered and reflected by the blood beneath the skin. The scattered and reflected light is indicated by the labelled region 60. The separation distance 65 between the laser module 20 and the image sensor 35 is at least twice or is about twice a penetration depth 70 of the light into the object. In other words, the ratio of the distance 65 between the laser module 20 and the image sensor 35 to a penetration depth 60 is at least 2:1 or is about 2:1. The penetration depth 70 required will depend on the depth of the object or parameter to be measured. For example, the separation distance 65 may be from 5 mm to 20 mm and will allow a penetration depth of approximately 2.5 mm to 10 mm. The skin is about 2 mm thick thus having the separation distance 65 from 5 mm to 20 mm in the sensing device 10 allows for the light to be emitted beyond the skin and into the deep tissue and blood vessels whereby it is scattered and reflected and measured by the image sensor 35.

When laser light is shone at a diffuse object it produces a random interference effect known as a speckle pattern. The speckles fluctuate in intensity when there is movement in the object. Hence, the sensing device 10 measures the changes in the laser speckle pattern to obtain data on the blood flow An example of the image sensor 35 is a CMOS sensor which captures a speckle pattern shown in FIG. 2. The pattern looks like grinding paper with a lot of small random dots. The dynamics of the scattering liquid may be extracted from this grinding paper pattern by calculating the statistical properties of the grinding paper pattern thereby allowing the laser speckle changes to be used as a proxy to measure the blood flow. A CCD sensor may also be used as the image sensor 35 without deviating from the embodiments described herein. Some generic setting changes in sensor sensitivity, integration time etc. may be needed to optimise the images.

An example of an image sensor 35 that may be used has a resolution of 1280×960 and measures 4.8 mm×3.6 mm. The image sensor 35 may have an imaging field at the centre of the image sensor 35. The imaging field may be from 7×7 pixels to 300×300 pixels. Thus, the size of the imaging field is relatively small compared to the size of the image sensor 35. In an example, a 20×20 pixels imaging field may be used and may have a small pixel size that is typically less than 3 microns while a 300×300 pixels imaging field will be about 1 mm×1 mm. Due to the relatively small size of the imaging field, there is no attenuation in the signal and the signal quality is the same. Furthermore, when the imaging field is in the centre of the image sensor 35, the effects of the changing intensity due to the distance travelled by the scattered and reflected light is minimised and the signal quality is consistent across the imaging field. The data from the imaging field may be used to determine the Dynamics Index (BPI).

The Dynamics index may be defined as:

$$Dynamics index = {( \frac{I}{S . D .} )}^{x} ,$$

where I is the mean intensity of all the pixels, S.D. is the standard deviation of all the pixels, and x is from a range of 1.8 to 2.6. The value of x as 2.2 is an optimal value from theory simulation performed.

The image sensor 35 may be configured to capture the light reflected from the object as a plurality of images (i.e. data from the imaging field) at a sampling rate of at least 100 Hz, preferably from 100 Hz to 500 Hz. Each of the images contain a plurality of pixels at each timepoint and the controller may be configured to determine a Dynamics index at each timepoint from each of the images.

The sensing device 10 does not require or use an optical conduit like an optical fibre to deliver light from the laser diode 26 to the object or collect light from the object to the image sensor 35, or a lens to converge or diverge the light.

The controller is integrally placed with the image sensor 35 and the miniaturized laser module 20 and the sensing device 10 can function on itself without requiring any remote controller or external computing device. A micro-SoM (system on module) or a FPGA (Field-programmable gate array) is engineered in the sensing device 10 itself to function as a controller. The controller may be configured to process a signal from the image sensor 35, the signal being related to the received light, which is explained in greater detail below.

Conventional diffuse correlation spectroscopy (DCS) requires calculation of autocorrelation function of the signals, and this takes on a significantly high computational load. In a multi-mode detection fibre-based method, diffuse speckle pulsatile flowmetry (DSPF) may generate a signal background (usually takes approximately 6,000 frames of speckle images) and this signal background may need to be removed from the detected signals. As a result, this poses a significant computing power requirement on the controller. By reducing all unnecessary computation, the microcontroller is able to perform all the necessary computations and actions on blood flow measurement and wireless communication in the sensing device 10 itself without the need for an external computation unit.

However, the imaging field size used for the image sensor 35 is sufficiently and relatively small that the signal from the image sensor 35 may be considered under or more specifically, along with the same signal background which may be negligible. Therefore, the background removal step may be omitted in the computation by the controller in the sensing device 10. Additionally, no autocorrelation of the detected signals is performed. Further, unlike some of the existing methods, the methods described herein do not require the calculation of the autocorrelation function of the detected signals and reduces the required computational power. Advantageously, the measurement methods described herein reduces the computational power required to process the raw data and allows a very small microcontroller unit (MCU) to be used as the processor. Advantageously, the computation of the Dynamics index and TVI may use simple mathematical operations like addition, subtraction, multiplication, division, square root etc and do not use matrix calculations required in autocorrelation and represents a significant change and improvement over existing methods and devices. The reduced computational power allows the embedded MCU to control the image sensor 35 and uses the raw measurements from the image sensor 35 to compute the Dynamics Index which is a general algorithm that measures the blood flow and converts it into a haemodynamic waveform. The Dynamics Index and haemodynamic waveform may be applied and used to assess the tissue viability, for example a Tissue Viability Index (TVI) is described in the subsequent paragraphs that provides a numerical and qualitative measurement of the tissue viability. This is unlike existing methods which are too computationally intensive to be done by an embedded MCU and require an external controller to obtain an equivalent value from the sensor.

### Application in Tissue Viability Assessment:

The existing method for tissue viability assessment is by the measurement of transcutaneous oxygen pressure (TcPO2). TcPO2 measures the oxygen pressure at a certain location of the tissue. If the oxygen pressure is high, then the tissue has a higher chance of healing. However, TcPO2 requires an expensive device to measure and a skilled professional to operate. This severely limits the measurement of TcPO2 to generally clinic settings and cannot be readily used by a user in daily activities.

When a plurality of dynamics indices is acquired at a high sampling rate (at least 100 Hz, for example from 100 to 500 Hz) from human body such as skin, a haemodynamic waveform may be obtained. Hence, the controller is configured to generate a plurality of the Dynamics Indices at different timepoints based on the sampling rate, generate a waveform based on the plurality of Dynamics Indices, and to convert the waveform into a frequency domain. Frequency domain analysis was applied to the haemodynamic waveform and a “Tissue Viability Index” is obtained to provide a qualitative assessment of the wound healing capability of the tissue. The haemodynamic waveforms from a healthy subject's foot and a diabetic patient with foot ulcer are shown in FIG. 8. The frequency domain analysis of the haemodynamic waveforms is also illustrated in FIG. 8. A clear peak around the heartbeat frequency (shown with the dashed box) may be seen in panel (b) of FIG. 4 for the healthy subject, but the peak (in the dashed box) in a diabetic patient is much less clear or distinct as shown in panel (d) of FIG. 8.

It has been found that the Tissue Viability Index (TVI) extracted from blood flow waveform have a strong correlation with the TcPO2 result. Hence, it is believed that the TVI obtained as described herein provides an easy and cheap way to replace TcPO2 measurement at least as an estimate. As the heart rate is approximately 1-2 Hz, when a sampling rate of the Dynamic Index is too slow or low, the detailed changes in each cardiac cycle cannot be measured and detected. Thus, a high sampling rate is necessary to obtain an accurate value of the TVI as the TVI requires high temporal resolution to perform the frequency domain signal analysis.

In the method used in the sensing device 10, the heartbeat frequency (which relates to the pulse rate) is used as a signal and the low frequency is used as a normalization factor to compensate for differences among different people. If the tissue pulsation is stronger, then this tissue has a higher chance to heal and stay healthy.

The Blood flow index is the blood flow waveform, shown in panel (a) of FIG. 5. The Tissue Viability Index (TVI) is an index that is extracted from the blood flow waveform. The blood flow waveform (acquired in time domain) is transformed into a frequency domain. Subsequently, the values of the low frequency band and heartbeat frequency band are extracted and their ratio calculated to get the TVI value.

The processor or controller may be programmed to control the image sensor 35 and calculate the TVI value by either of the methods shown below with minimal difference. In other words, the TVI may be determined by the controller of the sensing device 10 based on the data collected by the image sensor 35. While the heartbeat and pulse rate are technically different, for the purposes herein they may be used interchangeably as the pulse rate is often used as a common proxy, or measurement, of the heartbeat.

As examples, two methods to compute the Tissue Viability Index is provided below to exemplify how the TVI may be obtained from the data of the image sensor. However, it will be appreciated that there may be other methods (for example other constants or including other mathematical functions) to perform the calculation to obtain the ratio or other parameters that may be derived from the data and which may a correlation with the clinical assessment result.

In the first method, the TVI may be a ratio of a sum of amplitude values at a heartbeat frequency range in the frequency domain to a sum of amplitude values at a baseline frequency range in the frequency domain.

$$TVI = 300 \times \frac{{I}_{heartbeat}}{{I}_{LF}} \qquad Example 1$$

where I_{heartbeat} is the summation of the amplitude frequencies between heartbeat frequency-0.1 Hz and heartbeat frequency+0.1 Hz (in other words±0.1 Hz of the heartbeat frequency) and I_{LF} is the summation of all the amplitudes between 0 to 0.5 Hz (e.g. as shown in panel b and panel d of FIG. 8). The 0 to 0.5 Hz range is lower than the physiological heartbeat and thus provides a suitable baseline. The summation of the amplitude frequencies over a range of ±0.1 Hz of the heartbeat frequency may assist to smoothen the data.

In a second method, the TVI may be a ratio of an amplitude of a pulse rate frequency to an amplitude of frequency zero, or a square root of the ratio of the amplitude of the pulse rate frequency to the amplitude of frequency zero.

$$TVI = 200 \times \sqrt{\frac{{I}_{pulse \_ rate}}{{I}_{0}}} \qquad Example 2$$

where I_{pulse_rate} is the amplitude of the pulse rate frequency and I₀ is the amplitude of the amplitude at frequency 0 (the DC component).

The controller may be configured to determine the ratios and specifically the TVI without removal of background noise in the signal.

For both examples, the TVI value range for healthy tissue should be greater than or equal to 53, for example from 53-90. If the TVI value is less than 53, it would most likely be diseased tissue. If the TVI value is less than 35, the patients would need medical treatment as soon as possible. As an example, healthy subjects may have a TVI value range of 53-90, and diabetic foot patients may have a TVI range of 10-53. When the TVI is great than 0 to less than 10, there is a high change that the tissue would not survive based on the preliminary small cohort data available. It will be appreciated that the key in both formulas provided as examples is the ratio, the other mathematical operator like the multiplication and square root functions and the constant merely provide the TVI value in a range that is easier for users to work with.

Fundamentally, this method provides a quantitative way to measure the ‘pulsation strength’ of the tissue. A stronger pulsation of the tissue may imply a higher survival chance of the tissue. The TVI provides a direct and qualitative measurement of the tissue viability and may be potentially used to assess the tissue viability in diabetic patients and patients with peripheral artery disease.

FIG. 9 shows how the sensing device 10 may be used. In the method 300, in block 305, the sensing device 10 is placed at the measurement location. In block 310, the imaging field at the centre of the image sensor 35 is used to obtain the data for calculation. In block 315, the Dynamics Index (and TVI) is calculated as described above. The controller (or a processor or microprocessor) may be used to calculate the Dynamics index and TVI and may transmit the index through the wireless module to the external device 55.

The sensing device 10 may be used in externally measuring blood flow by emitting light at an object with blood flow beneath a surface of the object, receiving the light reflected from the object, and processing a signal relating to the received light. The reflected light from the object may be captured by the image sensor 35 as a plurality of images at a sampling rate of at least 100 Hz, preferably from 100 Hz to 500 Hz. The images may be used by controller to determine a Dynamics index at each timepoint. A plurality of the Dynamic indices at different timepoints based on the sampling rate may be generated which is used to generate a waveform based on the plurality of Dynamics indices. The waveform may then be converted into a frequency domain. The ratio or TVI may be computed as described above and preferably without removal of background noise of the signal and autocorrelation of the detected signals.

The sensing device 10 does not require or use a separate console machine to perform the calculations of the Dynamic Index and TVI but may be connected to an external device 55 like a laptop, a mobile device, a tablet, or other minicomputers to display the data (like the computed ratio or an indication based on the ratio) for easier viewing by a user. The simplification of the calculation and removal of several computation intensive processes means that a microcontroller within the sensing device 10 itself is sufficient to compute and provide the Dynamics Index and Tissue Viability Index to a user who can monitor their physical wellbeing directly with the sensing device 10 or more likely in the form of the wearable device without requiring a specialised and expensive machine that is likely only available in a clinical setting. For example, the indication may inform the user that the tissue is normal or healthy, or that the user should seek medical attention. The external device 55 may also be used for more detailed analysis of the data from the sensing device 10. For example, FIG. 1 shows a system 100 with the sensing device 10 connected to an external device 55 via a cable 50. Alternatively, the sensing device 10 may be connected wirelessly to the external device 55, for example via Bluetooth or Wi-Fi. Hence, the sensing device 10 may be provided with a transmitter, a receiver, and a transceiver. The transmitter, the receiver, and/or the transceiver is coupled to the controller and is configured to communicate wirelessly with an external device 55. The transmitter, the receiver, and/or the transceiver may be contained within a wireless module.

1. A sensing device comprising:
a single printed circuit board;
one or more laser diodes configured to emit light at an object;
one or more laser drivers configured to supply power to the one or more laser diodes;
an image sensor configured to receive and capture the light reflected and/or scattered from the object as a plurality of images at a sampling rate of at least 100 Hz, preferably from 100 Hz to 500 Hz; and
a controller configured to process a signal from the image sensor, the signal being related to the received light, wherein the one or more laser diodes, the one or more laser drivers, the image sensor, and the controller are integrally disposed on the single printed circuit board,
wherein each of the plurality of images comprises a plurality of pixels at each timepoint, the controller being configured to determine a Dynamics index at each timepoint, wherein the Dynamics index is calculated as
$${( \frac{I}{S . D .} )}^{x} ,$$
wherein I is the mean intensity of the pixels, S.D. is the standard deviation of the plurality of pixels, and x is from 1.8 to 2.6, preferably x is 2.2.

2. The sensing device according to claim 1, wherein the image sensor comprises a complementary metal-oxide-semiconductor (CMOS) sensor or a charge-couple device (CCD) sensor, and/or wherein the image sensor is configured to operate without an optical conduit to receive the light reflected and/or scattered from the object, and/or wherein the signal comprises a plurality of laser speckle patterns, and/or wherein the controller is configured to process the signal to measure changes in the plurality of laser speckle patterns, and preferably the controller comprises a micro system on module or a field-programmable gate array.

3-7. (canceled)

8. The sensing device according to claim 1, wherein the plurality of pixels comprises an imaging field at a center region of the image sensor, the imaging field having a range from 7×7 pixels to 300×300 pixels.

9. The sensing device according to claim 1, wherein the controller is configured to generate a plurality of the Dynamics indices at different timepoints based on the sampling rate; to generate a waveform based on the plurality of Dynamics indices; and to convert the waveform into a frequency domain, and/or wherein the controller is configured to determine the Dynamics index without removal of background noise in the signal.

10. The sensing device according to claim 9, wherein the controller is configured to determine a ratio of a sum of amplitude values at a heartbeat frequency range in the frequency domain to a sum of amplitude values at a baseline frequency range in the frequency domain.

11. The sensing device according to claim 10, wherein the heartbeat frequency range comprises from a heartbeat frequency minus 0.1 Hz to the heartbeat frequency plus 0.1 Hz and the baseline frequency range is from 0 Hz to 0.5 Hz.

12. The sensing device according to claim 9, wherein the controller is configured to determine a ratio of an amplitude of a pulse rate frequency to an amplitude of frequency zero, or a square root of the ratio of the amplitude of the pulse rate frequency to the amplitude of frequency zero.

13-14. (canceled)

15. The sensing device according claim 1, wherein a distance between the one or more laser diodes and the image sensor is at least twice a penetration depth of the light into the object.

16. The sensing device according to claim 1, further comprising at least one of the following: a display unit configured to display the ratio, or an indication based on the ratio, a transmitter, a receiver, a transceiver, and a power unit configured to provide electrical power to the sensing device, wherein the transmitter, the receiver, and/or the transceiver is coupled to the controller and is configured to communicate wirelessly with an external device.

17-18. (canceled)

19. The sensing device according to claim 1, wherein the one or more laser diodes each operates at a low optical power of 1 mW to 10 mW and the one or more laser drivers each is a constant current source, wherein an operating wavelength of each of the one or more laser diodes is from 450 nm to 1300 nm, preferably from 700 nm to 950 nm, and wherein each of the one or more laser diodes has a narrow linewidth of less than 0.1 mm.

20-21. (canceled)

22. A wearable device comprising the sensing device according to claim 1; and a fastening mechanism adapted to couple the sensing device to a user, wherein the wearable device comprises a watch, a wristband, or a skin patch to be pasted on a skin of the user.

23. (canceled)

24. A method of externally measuring blood flow, the method comprising:
emitting light at an object with blood flow beneath a surface of the object;
receiving and capturing the light reflected and/or scattered from the object as a plurality of images at a sampling rate of at least 100 Hz, preferably from 100 Hz to 500 Hz; and
processing a signal relating to the received light,
wherein each of the plurality of images comprises a plurality of pixels at each timepoint, and wherein the method further comprises determining a Dynamics index at each timepoint, wherein the Dynamics index is calculated as
$${( \frac{I}{S . D .} )}^{x} ,$$
I being the mean intensity of the pixels, S.D. being the standard deviation of the plurality of pixels, and x being from 1.8 to 2.6, preferably x being 2.2.

25-26. (canceled)

27. The method according to claim 24, further comprising generating a plurality of the Dynamic indices at different timepoints based on the sampling rate; generating a waveform based on the plurality of Dynamics indices; and converting the waveform into a frequency domain, and/or determining the Dynamics index is performed without removal of background noise of the signal.

28. The method according to claim 27, further comprising determining a ratio of a sum of amplitude values at a heartbeat frequency range in the frequency domain to a sum of amplitude values at a baseline frequency range in the frequency domain.

29. The method according to claim 28, wherein the heartbeat frequency range comprises from a heartbeat frequency minus 0.1 Hz to the heartbeat frequency plus 0.1 Hz and preferably the baseline frequency range is from 0 to 0.5 Hz.

30. The method according to claim 27, further comprising determining a ratio of an amplitude of a pulse rate frequency to an amplitude of frequency zero, or a square root of the ratio of the amplitude of the pulse rate frequency to the amplitude of frequency zero.

31. (canceled)

32. The method according to claim 24, wherein the method is performed by the sensing device according to of claim 1 or the wearable device according to claim 22.

33. A non-transitory computer readable medium comprising instructions that, when executed on a processor, perform the method according to claim 24.

34-35. (canceled)

36. A method of determining usability of a laser module in haemodynamic waveform measurement, the method comprising:
emitting light from the laser module at a rough surface of a metal;
receiving the light reflected from the rough surface to capture a plurality of images over a period of time, wherein each of the plurality of images comprises a plurality of pixels at each timepoint; and
determining a Dynamics index at each timepoint over the period of time, wherein the Dynamics index is calculated as
$${( \frac{I}{S . D .} )}^{x} ,$$
wherein I is the mean intensity of the pixels, S.D. is the standard deviation of the plurality of pixels, and x is from 1.8 to 2.6, preferably x is 2.2.

37. The method according to claim 36, further comprising determining the laser module to be usable when the Dynamic index is determined to be from 0 to 20 with a standard deviation of less than 2.
